SEQUENCE LISTING (8 sequences)

SEQ ID NO: 1 - protein, 30 aa, Homo sapiens
EQLKAISTRDPLSEITKQEKDFLWSHRHYC

SEQ ID NO: 2 - protein, 30 aa, Homo sapiens
EQLKAISTRDPLSEITEQEKDFLWSHRHYC

SEQ ID NO: 3 - protein, 18 aa, Homo sapiens
SEITKQEKDFLWSHRHYC

SEQ ID NO: 4 - protein, 21 aa, Homo sapiens
EQLKAISTRDPLSEITKQEKD

SEQ ID NO: 5 - protein, 18 aa, Homo sapiens
SEITEQEKDFLWSHRHYC

SEQ ID NO: 6 - protein, 18 aa, Homo sapiens
  MOD_RES (9)..(9): X is stapled (s)-N-Fmoc-2-(4′-pentenyl) alanine
SEITEQEKXFLWXHRHYC

SEQ ID NO: 7 - protein, 18 aa, Homo sapiens
SEITEQEKDFLWSHRHYC

SEQ ID NO: 8 - protein, 18 aa, Homo sapiens
  MOD_RES (9)..(9): X is stapled (s)-N-Fmoc-2-(4′-pentenyl) alanine
SEITEQEKXFLWXHRHYC

(19) United States
(12) Patent Application Publication
(10) Pub. No.: US 2014/0005119 A1
(43) Pub. Date: Jan. 2, 2014

(54) COMPOSITIONS AND METHODS FOR INHIBITING THE ACTIVITY OF P110a MUTANT PROTEINS

(71) Applicant: Case Western Reserve University, Cleveland, OH (US)

(72) Inventors: Zhengne Wang, Beachwood, OH (US); Yujun Hao, Cleveland Heights, OH (US); Chao Wang, Singapore (SG); Weiping Zheng, Zhenjiang City (CN)

(21) Appl. No.: 13/931,263

(22) Filed: Jun. 28, 2013

Related U.S. Application Data
(60) Provisional application No. 61/665,760, filed on Jun. 28, 2012.

(51) Int. Cl. C12N 9/12

(52) U.S. Cl. CPC C12N 9/1205
USPC 514/19.3; 435/184

(57) ABSTRACT

A method of inhibiting the activity, signaling, and/or function of a p110α mutant protein in a cancer cell expressing the p110α mutant protein includes administering to the cancer cell an amount of a therapeutic agent effective to inhibit binding of the p110α mutant protein to IRS1 in the cell.

### RELATED APPLICATION

This application claims priority from U.S. Provisional Application No. 61/665,760, filed Jun. 28, 2012, the subject matter of which is incorporated herein by reference in its entirety.

### TECHNICAL FIELD

This application relates to compositions and methods for inhibiting or reducing the activity, signaling and/or function of p110α mutant proteins and to methods and compositions for treating diseases, disorders, and/or conditions associated with the activity, signaling, and/or function of p110α mutant proteins in cancer and tumor cells.

### BACKGROUND

The PIK3CA gene encodes the catalytic subunit of the enzymatic polypeptide phosphatidylinositol 3-kinase α (PI3Kα), which plays a key role in regulating cell proliferation, survival and motility. PI3Kαconsists of a catalytic subunit p110α and one of several regulatory subunits, a major one being p85α. The p110α subunit contains an N-terminal adaptor-binding domain (ABD), a Ras binding domain (RBD), a C2 domain, a helical domain and a catalytic domain. At the basal state, the regulatory p85 subunit stabilizes the catalytic p110α subunit and inhibits its enzymatic activity. Upon growth factor stimulations, the SH2 domains of p85 bind to the phosphotyrosine residues on the receptor protein kinases or adaptor proteins such as insulin receptor substrate 1 (IRS1), thereby activating the lipid kinase activity of PI3Kα. Activated PI3Kαconverts phosphatidylinositol-4,5-bisphosphate (PIP2) to phosphatidylinositol-3,4,5-triphosphate (PIP3). The second messenger PIP3 then activates downstream AKT signaling. AKT is associated with tumor cell survival, proliferation, and invasiveness in many cancers.

PIK3CA is frequently mutated in a variety of human cancers including colorectal cancers (CRCs). Most of p110α mutations occur at two hot spots: the E545K mutation in the helical domain and the H1047R mutation in the kinase domain. It has been proposed that the E545K and H1047R mutations exert their oncogenic functions through distinct mechanisms. Structural analysis indicates that the p110α H1047R mutation alters the interaction between PI3Kα and cell membrane, thereby activating its kinase activity. It has also been proposed that the helical domain mutations, such as E545K mutations, activate their enzymatic activities by disrupting the inhibitory effect of the p85 subunit. This hypothesis is based on an in vitro biochemical analysis. However, cellular evidence revealing the mechanism by which a p110α E545K mutation exerts its oncogenic functions remains to be observed.

### SUMMARY

Embodiments described herein relate to a method of inhibiting the activity, signaling, and/or function of a p110α mutant protein in a cancer cell expressing the p110α mutant protein. The method includes administering to the cancer cell an amount of a therapeutic agent effective to inhibit binding of the p110α mutant protein to IRS1 in the cell. The therapeutic agent includes a polypeptide consisting of about 10 to about 40 amino acids. The polypeptide can have at 80% sequence identity with consecutive amino acids of a portion of a helical domain of the p110α mutant protein that includes the mutated amino acid or domain. Inhibition of binding of the p110α mutant protein to IRS1 can thereby inhibit the catalytic activity, signaling, and function of the p110α mutant protein in the cancer cell. The amount of the therapeutic agent administered to the subject can be effective to inhibit proliferation, survival and/or motility of the cancer cell.

In some embodiments, the p110α mutant protein can include a p110α helical domain mutant protein selected from the group consisting of an E545K, E542K, E545A, E545G or Q546K p110α helical domain mutant protein. In other embodiments, the therapeutic agent can bind to or complex with a portion of IRS1 corresponding to AA 585-962. By way of example, the polypeptide can have an amino acid sequence selected from the group consisting of SEQ ID NOs: 1, 3, 4, 7 and 8. In still other embodiments, the therapeutic agent can include a stapled macrocyclic derivative of the polypeptide.

In some embodiments, the cancer cell can include a solid tumor cell selected from the group consisting of a colon cancer cell, a rectal cancer cell, a lung cancer cell, a brain cancer cell, a head & neck cancer cell, a breast cancer cell, a skin cancer cell, a liver cancer cell, a pancreatic cancer cell, a stomach cancer cell, a uterine cancer cell, a cervical cancer cell, an ovarian cancer cell, a testicular cancer cell, a skin cancer cell or a esophageal cancer cell.

Other embodiments described herein relate to a method of treating cancer in a subject. The cancer includes cancer cells expressing a p110α mutant protein. The method includes administering to the cancer cells an amount of a therapeutic agent effective to inhibit binding of the p110α mutant protein to IRS1 in the cell. The therapeutic agent includes a polypeptide consisting of about 10 to about 40 amino acids. The polypeptide can have at 80% sequence identity with consecutive amino acids of a portion of a helical domain of the p110α mutant protein that includes the mutated amino acid. Inhibition of binding of the p110α mutant protein to IRS1 can thereby inhibit the catalytic activity, signaling, and function of the p110α mutant protein in the cancer cell. The amount of the therapeutic agent administered to the subject can be effective to inhibit proliferation, survival and/or motility of the cancer cell.

In some embodiments, the p110α mutant protein can include a p110α helical domain mutant protein selected from the group consisting of an E545K, E542K, E545A, E545G or Q546K p110α helical domain mutant protein. In other embodiments, the therapeutic agent can bind to or complex with a portion of IRS1 corresponding to AA 585-962. By way of example, the polypeptide can have an amino acid sequence selected from the group consisting of SEQ ID NOs: 1, 3, 4, 7 and 8. In still other embodiments, the therapeutic agent can include a stapled macrocyclic derivative of the polypeptide.

In some embodiments, the cancer cell can include a solid tumor cell selected from the group consisting of a colon cancer cell, a rectal cancer cell, a lung cancer cell, a brain cancer cell, a head & neck cancer cell, a breast cancer cell, a skin cancer cell, a liver cancer cell, a pancreatic cancer cell, a stomach cancer cell, a uterine cancer cell, a cervical cancer cell, an ovarian cancer cell, a testicular cancer cell, a skin cancer cell or a esophageal cancer cell.

In yet another aspect, the invention relates to a pharmaceutical composition. The composition includes a therapeutic agent and a pharmaceutically acceptable carrier, the therapeutic agent inhibiting binding of a p110α mutant protein to IRS1 when administered to a cancer cell expressing the p110α mutant protein. The therapeutic agent can include a polypeptide consisting of about 10 to about 40 amino acids. The polypeptide can have at 80% sequence identity with consecutive amino acids of a portion of a helical domain of the p110α mutant protein that includes the mutated amino acid. By way of example, the polypeptide can have an amino acid sequence selected from the group consisting of SEQ ID NOs: 1, 3, 4, 7 and 8. In still other embodiments, the therapeutic agent can include a stapled macrocyclic derivative of the polypeptide

### BRIEF DESCRIPTION OF THE DRAWINGS

FIGS. 1(A-H) illustrate: (A, C, E, and G) Schematic diagrams of IRS1 deletion proteins. (B) Immunoblots of His-tagged IRS1 constructs as shown in (A) incubated with lysates made from either the p110α mutant-only or WT-only DLD1 cells and blotted with an anti-p110α antibody. (D) Immunoblots of recombinant 6×His tagged IRS1 proteins shown in (C) incubated with cell lysates made from the p110α mutant-only DLD1 cells and blotted with an anti-p110α antibody. (F) Immunoblots of GST-fusion proteins shown in (E) incubated with lysates made from the p110α mutant-only DLD1 cells and blotted with an anti-p110α antibody. (H) Immunoblots of MYC-tagged full-length IRS1 or the IRS1D construct cotransfected with either a WT p110α plasmid or a p110α E545K mutant plasmid and a HA-tagged p85 plasmid and blotted with the indicated antibodies.

FIGS. 2(A-C) illustrate: (A) A plot showing three million cells of the indicated genotypes were injected subcutaneously into athymic nude mice. Tumor sizes were measured weekly for 5 weeks starting 2 weeks after injection. (B) Immunoblot of cell lysates of two stable clones of IRS1 KO DLD1 cells reconstituted with either full-length IRS1 or IRS1D. (C) Immunoblots and plots of tumor volume of xenograft of the indicated cell lines established as described in (A). Tumor sizes were measured weekly for 5 weeks. Mice were then sacrificed and tumors were harvested and photographed. The p value was calculated using ANOVA analysis. All quantitative measurements are plotted as mean±SEM.

FIGS. 3(A-M) illustrate: (A, B) Mapping peptides (P3, SEQ ID NO: 1; P4 SEQ ID NO: 2; P5, SEQ ID NO: 3; P6, SEQ ID NO: 4) derived from mutant p110α that inhibit interaction between IRS1 and p110α E545K. The indicated peptides were added into cell lysates made from the p110α E545K 3×FLAG KI cells. Cell lysates were immunoprecipitated with beads conjugated with anti-FLAG antibodies and then blotted with anti-IRS1 antibodies. (C) Immunoblots of cell lysates made from the p110α E545K 3×FLAG KI cells. Cell lysates were immunoprecipitated with beads conjugated with anti-FLAG antibodies and then blotted with the indicated antibodies. (D) Immunoblots of cell lysate of Myc-tagged IRS1 plasmid, which co-transfected an ABD domain deletion construct of the FLAG-tagged p110α E545K mutant (ΔABD), incubated with the indicated amount of stapled p110α mutant peptides, and then immunoprecipitated with anti-FLAG antibodies and blotted with the indicated antibodies. (E) Graphs showing quantification of Western blots shown in FIG. 4B. (F) Quantification of Western blots shown in FIG. 4C. (G) Graphs showing quantification of Western blots shown in FIG. 4D. (H) Quantification of Western blots shown in FIG. 6E. (I) Quantification of Western blots shown in FIG. 4F. (J, K) Immunoblot and graph of purified recombinant p110α E545K mutant proteins mixed with the indicated components and quantified using an in vitro lipid kinase assay. (L, M) Immunoblots of the indicated cell lines serum-starved, stimulated with 1 μg/ml insulin for 30 min, and then treated with or without the indicated peptides. Cell lysates were blotted with the indicated antibodies. All error bars show mean±SEM. *p<0.05, **p<0.001, t test.

FIG. 4(A-F) illustrate: (A) Schematic diagram of linear and stapled WT and mutant peptides derived from p110α (SEQ ID NOs: 5, 6, 7 and 8). (B) Immunoblots of cell lysates immunoprecipitated with beads conjugated with anti-FLAG antibodies and then blotted with the indicated antibodies. Linear or stapled WT and mutant peptide were added into culture medium of the p110α E545K 3×FLAG KI cells for 16 hr. (C) Immunoblots of cell lysates immunoprecipitated with anti-p110α antibodies and then blotted with the indicated antibodies. Vaco481 CRC cells were treated with the stapled mutant peptides at the indicated concentration. (D) Immunoblots of cell lysates blotted with the indicated antibodies. The indicated cell lines were treated with the stapled mutant peptide. (E) Immunoblots of cell lysates blotted with the indicated antibodies. The indicated cancer cell lines were treated or untreated with stapled WT or mutant peptide at 50 mM for 2 hr. (F) Immunoblots of cell lysates were blotted with the indicated antibodies. The indicated cancer cell lines were treated with or without 50 mM of stapled mutant peptide for 2 hr.

FIGS. 5(A-D) illustrate: (A) An athymic nude mouse with a xenograft Tumor growth of cancer cells harboring the p110α E545K Mutation (A and B) and treated with Stapled p110α E545K Mutant Peptide. Five million DLD1 cells were injected subcutaneously and bilaterally into athymic nude mice. Once the xenograft tumors reach 100-150 mm³, the left side tumors were injected with the stapled WT peptide and the right side tumors were injected with the stapled mutant peptide daily for 14 days. Ten tumors of similar size in five other athymic nude mice were injected with an equal amount of water as controls. Tumor sizes were measured every 2 days. Representative pictures of mice injected with the peptides are shown in (A) and quantitative measurements are shown in (B). Arrows indicate tumors treated with the stapled WT peptide and the red arrows indicate tumors treated with the stapled mutant peptide. The p value was calculated using ANOVA analysis. (C and D) Identical experiments as described above were performed with HCT116 cells. All quantitative measurements are plotted as mean±SEM.

FIG. 6 is an immunoblot illustrating the stapled mutant peptide inhibits AKT signaling in xenograft tumors.

FIGS. 7(A-B) illustrate schematic diagrams of a model of growth factor independent Activation of the p110α E545K Mutant Signaling Pathway (A) When normal cells are stimulated by growth factors, p110a is brought to cell membrane through binding of p85 to phospho-IRS1, whereby converting PIP2 to PIP3. (B) In cancer cells harboring a p110a-helical domain mutation, the p110a mutant directly binds to IRS1, thereby being recruited to the cell membrane and converting PIP2 to PIP3.

### DETAILED DESCRIPTION

The embodiments described herein are not limited to the particular methodology, protocols, and reagents, etc., and as such may vary. The terminology used herein is for the purpose of describing particular embodiments only, and is not intended to limit the scope of the present invention, which is defined solely by the claims. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

As used herein, “one or more of a, b, and c” means a, b, c, ab, ac, bc, or abc. The use of “or” herein is the inclusive or.

As used herein, the terms “subject” or “patient” or “animal” or “host” refer to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like). A patient refers to a subject afflicted with a disease or disorder (e.g., cancer). In some aspects of the disclosed methods, the subject has been diagnosed with a need for treatment of one or more neoplastic disorders prior to the administering step. In some aspects of the disclosed method, the subject has been diagnosed with a need for the inhibition of p110α mutant protein enzyme activity prior to the administering step. In some aspects, a p110α mutant protein associated with cancer has been detected in the cancer cells of the subject.

The agents, compounds, compositions, antibodies, etc. used in the methods of the invention are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence.

As used herein, “treating” or “treatment” of a condition may refer to preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition' or some combination thereof. With regard to neoplastic disorders, “treating” or “treatment” may refer to inhibiting or slowing neoplastic and/or malignant cell growth, proliferation, and/or metastasis, preventing or delaying the development of neoplastic and/or malignant cell growth, proliferation, and/or metastasis, or some combination thereof. With regard to a tumor, “treating” or “treatment” may refer to eradicating all or part of a tumor, inhibiting or slowing tumor growth and metastasis, preventing or delaying the development of a tumor, or some combination thereof.

As used herein, an “effective amount” of an agent is an amount sufficient to achieve a desired therapeutic or pharmacological effect, such as an amount that is capable of inhibiting the growth, proliferation, survival and/or motility of tumor or cancer cells. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

As used herein, a “therapeutically effective amount” refers to an amount effective, at dosages and for periods of time necessary, to achieve the desired therapeutic result. A therapeutic result may be, e.g., lessening of symptoms, reduction or cessation of tumor growth, prolonged survival, improved mobility, delayed metastasis and the like. A therapeutic result need not be a “cure.”

As used herein, the terms “neoplastic cell”, “cancer cell” or “tumor cell” refer to cells that divide at an abnormal (i.e., increased) rate. A neoplastic cell or neoplasm (tumor) can be benign, potentially malignant, or malignant.

As used herein, “administering” to a patient includes dispensing, delivering or applying an active compound in a pharmaceutical formulation to a subject by any suitable route for delivery of the active compound to the desired location in the subject (e.g., to thereby contact a desired cell such as a desired tumor or cancer cell), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route.

As used herein, the phrases “parenteral administration” and “administered parenterally” refer to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the phrases “systemic administration,” “administered systemically,” “peripheral administration” and “administered peripherally” mean the administration of a compound, drug or other material other than directly into a target tissue (e.g., a tumor site), such that it enters the animal's system and, thus, is subject to metabolism and other like processes, for example, subcutaneous administration.

As used herein, the term “antibody”, includes human and animal mAbs, and preparations of polyclonal antibodies, synthetic antibodies, including recombinant antibodies (antisera), chimeric antibodies, including humanized antibodies, anti-idiotopic antibodies and derivatives thereof. A portion or fragment of an antibody refers to a region of an antibody that retains at least part of its ability (binding specificity and affinity) to bind to a specified epitope. The term “epitope” or “antigenic determinant” refers to a site on an antigen to which antibody paratope binds. Epitopes formed from contiguous amino acids are typically retained on exposure to denaturing solvents whereas epitopes formed by tertiary folding are typically lost on treatment with denaturing solvents. An epitope typically includes at least 3, at least 5, or 8 to 10, or about 13 to 15 amino acids in a unique spatial conformation. Methods of determining spatial conformation of epitopes include, for example, x-ray crystallography and 2-dimensional nuclear magnetic resonance. See, e.g., 66 EPITOPE MAPPING PROTOCOLS IN METS. IN MOLECULAR BIO. (Morris, ed., 1996); Burke et al., 170 J. Inf. Dis. 1110-19 (1994); Tigges et al., 156 J. Immunol. 3901-10).

As used herein, terms “peptide” or “polypeptide”, refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, N.Y., 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green & amp; Wuts, PROTECTING GROUPS IN ORGANIC SYNTHESIS (John Wiley & amp; Sons, 1991). The functional groups of a peptide can also be derivatized (e.g., alkylated) using art-known methods.

Peptides can be synthesized and assembled into libraries comprising a few too many discrete molecular species. Such libraries can be prepared using well-known methods of combinatorial chemistry, and can be screened as described herein or using other suitable methods to determine if the library comprises peptides which can antagonize p110α mutant protein-IRS1 interaction. Such peptide antagonists can then be isolated by suitable means.

As used herein, the terms “portion”, “fragment”, “variant”, “derivative” and “analog”, when referring to a therapeutic polypeptide of the present invention, include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

As used herein, the term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of normatural amino acids).

As used herein, the terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

As used herein, the term “small molecule” refers to a low molecular weight organic compound, which is by definition not a polymer. The small molecule can bind with high affinity to a biopolymer, such as protein (e.g., IRS1), nucleic acid, or polysaccharide and in some instances alter the activity or function of the biopolymer. The upper molecular weight limit for a small molecule is about 800 Daltons, which allows for the possibility to rapidly diffuse across cell membranes so that they can reach intracellular sites of action. In addition, this molecular weight cutoff is a necessary but insufficient condition for oral bioavailability.

As used herein, the term “expression” or “expressing”, as in cancer cells expressing a p110α polypeptide mutation or a p110α mutant protein, refers to the process by which a polynucleotide sequence undergoes successful transcription and translation such that detectable levels of the amino acid sequence or protein are expressed.

As used herein, the terms “p110α mutant polypeptide” and “p110α mutant protein” are used interchangeably and refer to a p110α polypeptide comprising at least one p110α mutation selected from a helical, ABD, C2 and/or kinase domain mutation. In some embodiments, a p110α mutant protein includes at least one p110α mutation selected from an E545K, E542K, E545A, E545G, Q546K, K111N, N345K, M1043L. Additional p110α mutant proteins can include at least one p110α mutation in the helical domain associated with cancer wherein the mutated protein gains interaction with IRS1 without growth factor stimulation. Certain exemplary p110α mutant proteins include but are not limited to, allelic variants, splice variants, derivative variants, substitution variants, deletion variants, and/or insertion variants, fusion polypeptides, orthologs, and interspecies homologs.

This application relates to compositions and methods for inhibiting and/or reducing the activity, signaling, and/or function of mutated proteins of the PI3Kαcatalytic subunit p110α and to methods and compositions for treating diseases, disorders, and/or conditions associated with the oncogenic activity, signaling, and/or function of p110α mutant proteins in cancer and tumor cells.

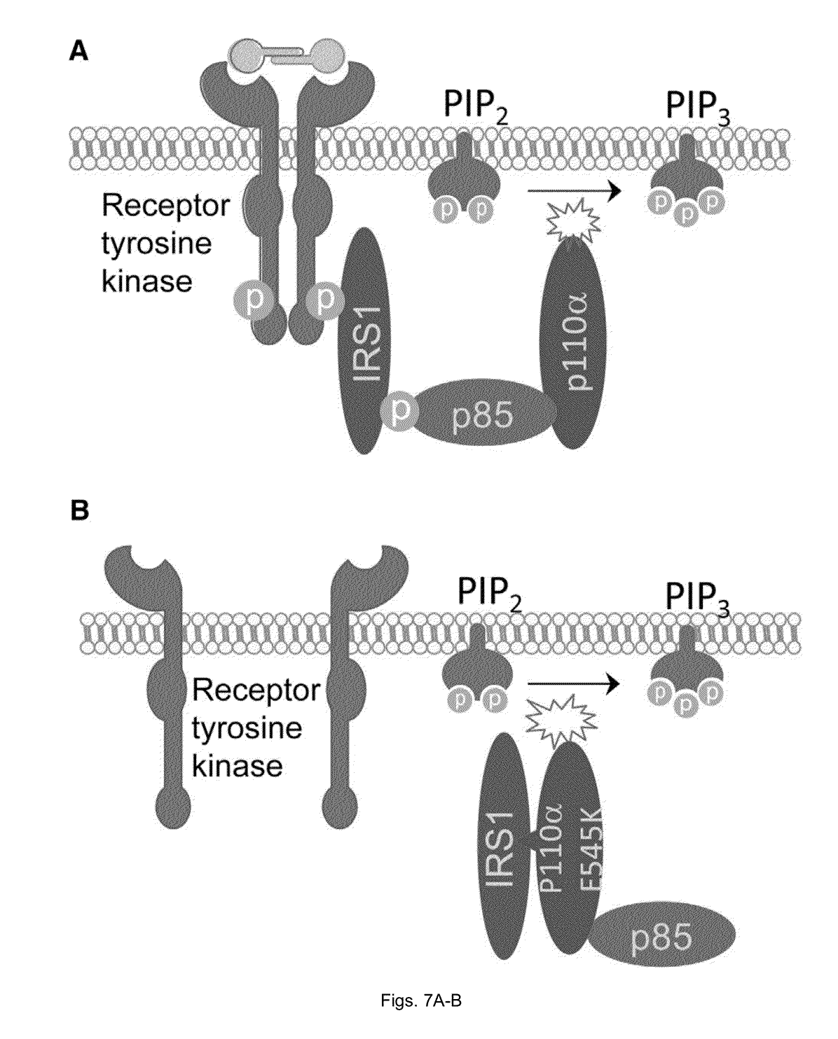

PI3Kαconsists of a catalytic subunit p110α and one of several regulatory subunits including p85α. Referring to FIG. 7A, at the basal state, the regulatory p85 subunit stabilizes the catalytic p110α subunit and inhibits its enzymatic activity. Upon growth factor stimulations, p85 binds to insulin receptor substrate 1 (IRS1), thereby activating the lipid kinase activity of PI3Kα and subsequent downstream AKT signaling.

As shown in FIG. 7B, it was found that even without growth factor stimulations, p110α mutant polypeptides in some cancer cells directly interact with IRS1 independently of the p85 regulatory subunit of PI3Kα and that this interaction is crucial for its oncogenic functions. It was also found that disruption of the IRS1-p110α mutant polypeptide specific interaction results in destabilization of p110α mutant protein, a reduction of downstream AKT phosphorylation, as well as the reduction of cell and tumor growth in cancer cells expressing a p110α polypeptide mutation. It was further found that agents that specifically inhibit the IRS1-p110α E545K mutant polypeptide interaction can inhibit and/or reduce tumor growth when injected into tumors with cells expressing a mutation in the helical domain of p110α. Accordingly, therapeutic agents that target and reduce or inhibit the specific interaction of IRS1 and a p110α mutant polypeptide in cancer cells that express a p110α mutant polypeptide mutation can be used to inhibit growth, proliferation, survival and motility of these cells.

Embodiments described herein can therefore relate to a method of inhibiting the activity, signaling, and/or function of a p110α mutant protein in a cancer cell. The method includes administering to the cancer cell an amount of a therapeutic agent effective to inhibit binding of the p110α mutant protein to IRS1 in the cell. The inhibition of binding of the p110α mutant protein to IRS1 thereby inhibits the catalytic activity, signaling, and function of the p110α mutant protein in these cells. Inhibiting binding of p110α mutant proteins to IRS1 can include completely or significantly disrupting IRS1 interaction with the p110α mutant proteins. Inhibiting and/or reducing the activity, signaling, and/or function of a p110α mutant protein in a cancer cell may also include inhibiting/reducing AKT phosphorylation related to p110α mutant-IRS1 binding in the cancer cells.

The amount of a therapeutic agent administered to a cancer cell may comprise the amount effective in inhibiting growth of a cancer cell, inhibiting proliferation of a cancer cell, inhibiting cancer cell survival, inhibiting cancer cell metastasis and/or reducing tumor burden in a subject.

The cancer cell expressing a p110α mutant protein may be a solid tumor cell, such as a lung cancer cell, a brain cancer cell, a head & neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a skin cancer cell or a esophageal cancer cell. In certain embodiments, cancer cells expressing a p110α mutant protein can include carcinoma cells such as colorectal, large cell cancer of the lung and breast carcinoma cells.

The therapeutic agent that inhibits binding of the p110α mutant protein to IRS1 in the cancer cell can include any agent that inhibits, blocks, decreases, suppresses and/or disrupts binding of one or more p110α mutant proteins to IRS1, thereby inhibiting the catalytic activity, signaling, and function of the p110α mutant protein in the cell. In some embodiments, the agent can include a competitive inhibitor or a dominant negative polypeptide. It is contemplated that such agents can be administered to a cancer cell or delivered intracellularly and once administered/delivered produce a destabilizing effect on the p110α mutant protein. The destabilizing effect can result in the decrease or reduction of downstream AKT phosphorylation as well as the decrease or reduction of cell growth, proliferation, survival and motility of these cancer cells expressing a p110α mutant protein.

Binding of a p110α mutant protein to IRS1 in a cancer cell can be inhibited, blocked, decreased, suppressed and/or disrupted by a therapeutic agent of the present invention in several ways including the use of a peptide/polypeptide, small molecule, a peptidomimetic, or an antibody. In one embodiment, the therapeutic agent that inhibits binding of an oncogenic p110α mutant protein to IRS1 in a cancer cell binds to and/or complexes with a portion of IRS1 corresponding to amino acid (AA) 585-962. In some embodiments, the therapeutic agent binds to and/or complexes with a portion of IRS1 corresponding to either AA 607-623 or AA 936-962. Alternatively, a therapeutic agent can include multiple inhibitory agents where a first agent binds to and/or complexes with a portion of IRS1 corresponding to AA 607-623 and a second agent binds to and/or complexes with a portion of IRS1 corresponding to AA 936-962.

In some embodiments, the therapeutic agent can include a polypeptide or a polypeptide mimetic having an amino acid sequence substantially homologous to a portion of a p110α mutant protein that includes a mutated domain or amino acid. The p110α subunit of PI3Kα contains an N-terminal adaptor-binding domain (ABD), a Rasbinding domain (RBD), a C2 domain, a helical domain and a catalytic domain. Immunoprecipitation analysis revealed that p110α mutant plasmids including E545K, E542K, E545A, E545G and Q546K mutations in the helical domain, a K111N mutation in the ABD domain, a N345K mutation in the C2 domain and a M1043L mutation in the kinase domain, gain interaction with IRS1 when compared to wild-type p110α subunit proteins. Therefore, a therapeutic agent may include a polypeptide that has an amino sequence that is substantially homologous to a portion thereof comprising at least one p110α mutation selected from a helical, ABD, C2 and/or kinase domain mutation, wherein the mutated protein gains interaction with IRS1 without growth factor stimulation. In certain embodiments, a therapeutic agent may include or a polypeptide mimetic having an amino acid sequence substantially homologous to a portion of a p110α mutant protein that includes a mutation selected from E545K, E542K, E545A, E545G, Q546, K111N, N345K and M1043L.

In some embodiments, a therapeutic agent may include a polypeptide or a polypeptide mimetic having an amino acid sequence substantially homologous to a portion of a p110α mutant protein comprising at least one p110α mutation in the helical domain associated with cancer wherein the mutated protein gains interaction with IRS1 without growth factor stimulation. Additional helical domain mutations of p110α that frequently occur in cancer include, but are not limited to E545Q, E545V, E542Q, E542V, Q546P, Q546R, Q546E, and Q546L. Therefore, it is further contemplated that a therapeutic agent may include a p110α polypeptide or a portion thereof comprising at least one p110α mutation selected from a p110α helical domain mutation selected from E545Q, E545V, E542Q, E542V, Q546P, Q546R, Q546E, and Q546L.

In other embodiments, therapeutic agent can include a polypeptide consisting of about 10 to about 40 amino acids that is substantially homologous to consecutive amino acids of a portion of a helical domain of the p110α mutant protein that includes the mutated amino acid or domain By substantially homologous, it is meant the peptide has at least about 80%, about 90%, about 95%, about 96%, about 97%, about 98%, about 99% or about 100% sequence identity with consecutive amino acids of a portion of the amino acid sequence of a mutated helical domain (e.g., a p110α E545K mutant helical domain).

Polypeptides and/or proteins described herein may also include, for example, biologically active mutants, variants, fragments, chimeras, and analogs; fragments encompass amino acid sequences having truncations of one or more amino acids, wherein the truncation may originate from the amino terminus (N-terminus), carboxy terminus (C-terminus), or from the interior of the protein. Analogs of the invention involve an insertion or a substitution of one or more amino acids. Variants, mutants, fragments, chimeras and analogues may function as inhibitors of p110α oncogenic mutant IRS1 interaction (without being restricted to the present examples).

Polypeptides derived from p110α that include an E545K helical domain mutation inhibit the interaction between a p110α E545K helical domain mutant protein and IRS1 proteins expressed in a cell. Therefore, in some embodiments, the therapeutic agent can include a polypeptide consisting of about 10 to about 40 amino acids that is substantially homologous (e.g., has at least 80% sequence identity) to about 10 to about 40 consecutive amino acids of the p110α E545K mutant helical domain. Example of a polypeptide that is substantially homologous to the p110α E545K mutant helical domain and can specifically bind to or complex with IRS1 can have an amino acid sequence of SEQ ID NO: 1, 3, 4, and 7.

Structural analysis has revealed that the p110α E545K mutant helical domain includes two α-helix subdomains (i.e., a C-terminal and a N-terminal α-helical motif) flanking the E545K mutation site. It was further revealed that a peptide corresponding to the C-terminal α-helical motif inhibited the interaction between p110α E545K mutant and IRS1 proteins by binding to IRS1. Therefore, in another embodiment, the therapeutic agent can include a polypeptide that is substantially homologous to about 15 to about 25 consecutive amino acids of C-terminal α-helical motif of the p110α E545K mutant helical domain.

One or more of, the peptides and proteins described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide.

A polypeptide described above can be modified by peptide stabilization techniques known in the art. Methods of stabilizing peptides for use as therapeutics can include stapling and cyclization of linear peptides. The general approach for “stapling” a peptide is that two key residues within the peptide are modified by attachment of linkers through the amino acid side chains. Once synthesized, the linkers are connected through a catalyst, thereby creating a bridge that physically constrains the peptide into its native a-helical shape. In addition to helping retain the native structure needed to interact with a target molecule, this conformation also provides stability against peptidases as well as cell-permeating properties. U.S. Pat. Nos. 7,192,713 and 7,183,059, describing this technology, are hereby incorporated by reference. Also see L. D. Walensky et al., Science 305, 1466 (2004), which is incorporated herein by reference.

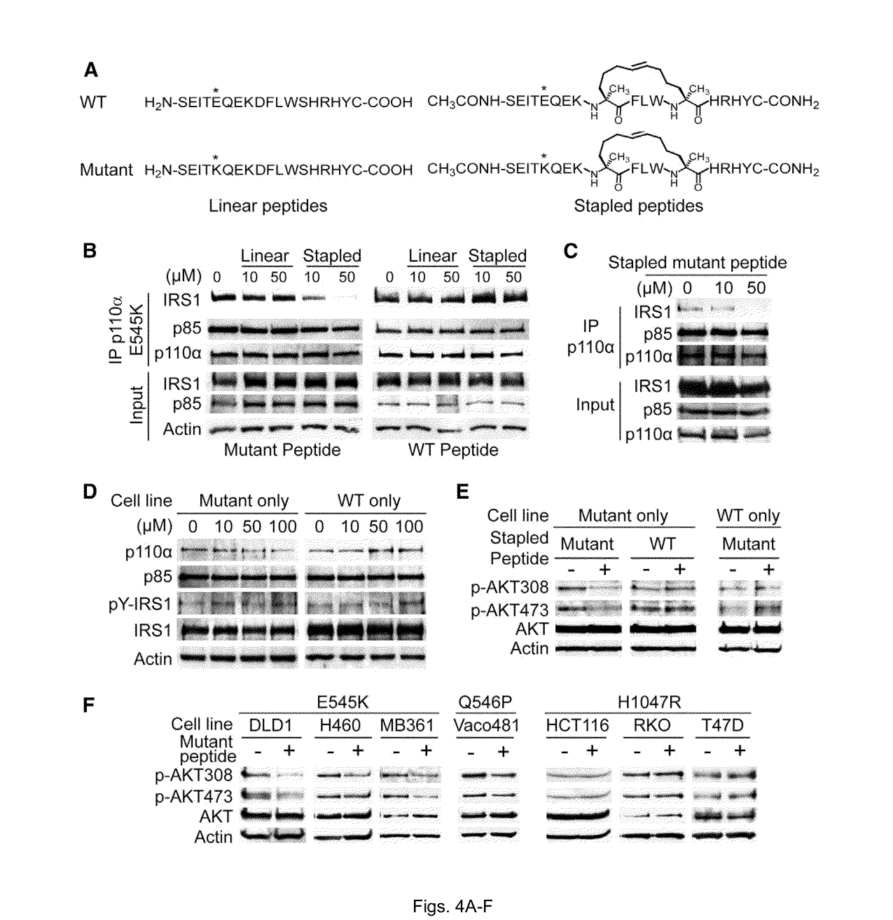

A linear polypeptide described herein can be stapled in order to stabilize one or more α-helical conformations of a linear peptide and thus enhance its binding affinity toward its target as well as increase cell-permeability. As shown in FIG. 4, a stapled polypeptide substantially homologous to the C-terminal α-helical motif of the p110α E545K mutant helical domain disrupted IRS1 interaction with a p110α E545K mutant in cell lysates and further reduced AKT phosphorylation in cancer cells.

Therefore, in some embodiments, the therapeutic agent can include a stapled macrocyclic derivative of a linear polypeptide substantially homologous to a p110α mutant protein or domain thereof. In one embodiment, the therapeutic agent can include a stapled macrocyclic derivative of a linear peptide substantially homologous to the C-terminal α-helical motif of the p110α E545K mutant helical domain. An example of a stapled peptide that is substantially homologous to the C-terminal α-helical motif of the p110α E545K mutant helical domain and can specifically bind to or complex with IRS1 can have an amino acid sequence of SEQ ID NO: 8 and is stapled at the D549 amino acid residue and the S553 residue as illustrated in FIG. 4A.

Additional modifications to the polypeptide can include, for example, without limitation, acetylation, acylation, addition of acetomidomethyl (Acm) group, ADP-ribosylation, amidation, covalent attachment to flavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Other type of peptide modifications may include for example, amino acid insertion (i.e., addition), deletion and substitution (i.e., replacement), either conservative or non-conservative (e.g., D-amino acids) in the polypeptide sequence where such changes do not substantially alter the overall competitive inhibitor ability of the polypeptide.

The polypeptides of this application may be prepared by methods known to those skilled in the art. The peptides may be prepared using recombinant DNA. For example, one preparation can include cultivating a host cell (bacterial or eukaryotic) under conditions, which provide for the expression of peptides and/or proteins within the cell.

The purification of the polypeptides and/or proteins may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

The therapeutic agents including the polypeptides and/or proteins described herein can also be in the form of or include a conjugate protein or drug delivery construct having at least a transport subdomain(s) or moiety(ies) (i.e., transport moieties). The transport moieties can facilitate uptake of the polypeptides into a mammalian (i.e., human or animal) tissue or cell (e.g., cancer cell). The transport moieties can be covalently linked to a peptides and/or proteins. The covalent link can include a peptide bond or a labile bond (e.g., a bond readily cleavable or subject to chemical change in the interior target cell environment). Additionally, the transport moieties can be cross-linked (e.g., chemically cross-linked, UV cross-linked) to the polypeptide.

The transport moieties can be repeated more than once in the polypeptides and/or proteins. The repetition of a transport moiety may affect (e.g., increase) the uptake of the peptides and/or proteins by a desired cell. The transport moiety may also be located either at the amino-terminal region of an active agent or at its carboxy-terminal region or at both regions.

In one embodiment, the transport moiety can include at least one transport peptide sequence that allows the polypeptides to penetrate into the cell by a receptor-independent mechanism. In one example the inhibitory peptide is a synthetic peptide that contains a Tat-mediated protein delivery sequence.

Other examples of known transport moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, (conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No.: 2,301,157. Similarly, HIV Tat protein was shown to be able to cross cellular membranes.

In addition, the transport moiety(ies) can include polypeptides having a basic amino acid rich region covalently linked to an active agent moiety (e.g., intracellular domain-containing fragments inhibitor peptide). As used herein, the term “basic amino acid rich region” relates to a region of a protein with a high content of the basic amino acids such as arginine, histidine, asparagine, glutamine, and lysine. A “basic amino acid rich region” may have, for example 15% or more of basic amino acid. In some instance, a “basic amino acid rich region” may have less than 15% of basic amino acids and still function as a transport agent region. In other instances, a basic amino acid region will have 30% or more of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of this application can function as a transport agent region.

In one embodiment, the polypeptides described herein can be non-covalently linked to a transduction agent. An example of a non-covalently linked polypeptide transduction agent is the Chariot protein delivery system (See U.S. Pat. No. 6,841,535; J Biol Chem 274(35):24941-24946; and Nature Biotec. 19:1173-1176, all herein incorporated by reference in their entirety).

The therapeutic agents described herein may further be modified (e.g., chemically modified). Such modification may be designed to facilitate manipulation or purification of the molecule, to increase solubility of the molecule, to facilitate administration, targeting to the desired location, to increase or decrease half life. A number of such modifications are known in the art and can be applied by the skilled practitioner.

A therapeutic agent described herein may be encapsulated or embedded in a delivery vehicle, such as a liposome, a lysosome, a microcapsule or a nanoparticle. In some cases, a polypeptide may be PEG-ylated.

Therapeutic agents described herein can be delivered by encapsulating or embedding in a delivery vehicle. For example, liposomes, which are artificially prepared vesicles made of lipid bilayers have been used to deliver a variety of drugs. Liposomes can be composed of naturally-derived phospholipids with mixed lipid chains (like egg phosphatidylethanolamine) or other surfactants. In particular, liposomes containing cationic or neutral lipids have been used in the formulation of drugs. Liposomes should not be confused with micelles and reverse micelles composed of monolayers, which also can be used for delivery.

Nanoparticles are generally considered to be particulate substances having a diameter of 100 nm or less. In contrast to liposomes, which are hollow, nanoparticles tend to be solid. Thus, the agent will be less entrapped and more either embedded in or coated on the nanoparticle. Nanoparticles can be made of metals including oxides, silica, polymers such as polymethyl methacrylate, and ceramics. Similarly, nanoshells are somewhat larger and encase the delivered substances with these same materials. Either nanoparticles or nanoshells permit sustained or controlled release of the peptide or mimetic, and can stabilize it to the effects of in vivo environment.

Another modification for delivery of peptides and peptidomimetics is PEG-ylation. PEG-ylation is the process of covalent attachment of polyethylene glycol polymer chains to another molecule, normally a drug or therapeutic protein. PEG-ylation is routinely achieved by incubation of a reactive derivative of PEG with the target macromolecule. The covalent attachment of PEG to a drug or therapeutic protein can “mask” the agent from the host's immune system (reduced immunogenicity and antigenicity), increase the hydrodynamic size (size in solution) of the agent which prolongs its circulatory time by reducing renal clearance. PEG-ylation can also provide water solubility to hydrophobic drugs and proteins. Exemplary PEG-ylation technologies are described in U.S. Pat. Nos. 7,666,400, 7,610,156, 7,587,286, 6,552,170 and 6,420,339.

Additional therapeutic agents for use in the present invention can be found by identifying potential agents that inhibit, block, decrease, suppresses and/or disrupt binding of one or more p110α mutant proteins to IRS1, thereby inhibiting the catalytic activity, signaling, and function of the p110α mutant protein in the cell. The ability of an agent to inhibit binding of a p110α mutation in a cancer cell may be assessed using any of a variety of known procedures and assays and/or disclosed within the instant application. Test compounds can be synthetic or naturally occurring. They can be previously identified to have physiological activity or not. Candidate compounds may be screened individually, in combination, or as a library of compounds.

Tests on candidate agents can be run in cell-free systems or in whole cells. p110α activity can be tested and measured by any means known in the art. Peptide competition assays may be employed using cancer cell lysates from cancer cells which harbor a p110α E545K mutation in order to evaluate the ability of an agent to competitively inhibit p110α mutant-IRS1 binding. For example, additional therapeutic agents can be identified as those agents having specificity for or that bind to a portion of IRS1 corresponding to amino acid (AA) 585-962 and thereby inhibit binding of p110α E545K mutant proteins to IRS1.

Tests on candidate agents can be conducted using animal models. Exemplary animal models suitable for use in the assays of the present invention includes the use of xenograft tumor models in mice where the mice are injected with DLD1 cells, which express a p110α E545K mutation. Once tumors reach a suitable size, administration to these animals of a potential therapeutic agent can be used to assess whether a given agent, route of administration, or dosage provides a therapeutic effect, such as decreasing cancer cell growth, proliferation, survival and/or motility in the test animals.

Candidate compounds screened include chemical compounds. In some aspects of the invention, the candidate compound is a small organic molecule having a molecular weight of more than about 50 and less than about 2,500 daltons. Compounds screened are also found among biomolecules including, but not limited to: peptides, saccharides, fatty acids, steroids, pheromones, purines, pyrimidines, derivatives, structural analogs or combinations thereof. The compounds screened can include functional groups necessary for structural interaction with proteins, particularly hydrogen bonding, and typically include at least an amine, carbonyl, hydroxyl, or carboxyl group.

Candidate compounds are obtained from a wide variety of sources including libraries of synthetic or natural compounds. Compounds to be screened can be produced, for example, by bacteria, yeast or other organisms (e.g., natural products), produced chemically (e.g., small molecules, including peptidomimetics), or produced recombinantly. It is further contemplated that natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

In many drug screening programs, with test libraries of compounds and natural extracts, high throughput assays are desirable in order to maximize the number of compounds surveyed in a given period of time. Assays of the present invention may be developed with purified or semi-purified proteins or with lysates. These assays are often preferred as “primary” screens in that they can be generated to permit rapid development and relatively easy detection of an alteration in a molecular target, which is mediated by a test agent. Assays of the present invention can include cell-based assays. Cell-based assays may be performed as either a primary screen, or as a secondary screen to confirm the activity of compounds identified in a cell free screen, such as an in silico screen.

For in vivo screening of candidate compounds, the candidate compound can be administered in any manner desired and/or appropriate for delivery of the compound in order to affect a desired result. For example, the candidate compound can be administered to a mammalian subject by injection (e.g., by injection intravenously, intramuscularly, subcutaneously, or directly into the cancer or tumor tissue in which the desired affect is to be achieved), topically, orally, or by any other desirable means.

Normally, this screen will involve a number of animals receiving varying amounts and concentrations of the candidate compounds (from no compound to an amount of compound that approaches an upper limit of the amount that can be delivered successfully to the animal), and may include delivery of the compound in different formulations. The compounds can be administered singly or can be combined in combinations of two or more, especially where administration of a combination of compounds may result in a synergistic effect.

The effect of compound administration upon the animal model can be monitored by any suitable method such as assessing the number and size of tumors, overall health, survival rate, etc. A candidate compound is identified as an effective compound for use in the treatment of a cancer in a subject where candidate compound inhibits cancer cell growth in the animal in a desirable manner (e.g., by binding to the IRS1 protein and competitively inhibiting p110α mutant-IRS1 binding, etc.). In some aspects, effective compounds can be identified as having low toxicity in vivo.

The discovery of frequent mutations of PIK3CA in human cancer provides a strong rationale for inhibiting p110α mutant protein activities as a targeted cancer therapy. Therefore, the present invention contemplates that disrupting the interactions between p110α mutant proteins expressed in cancer cells and IRS1 can be exploited as a targeted therapy for cancer patients with cancer cells harboring these mutations.

As shown in the Example below, the p110α E545K mutant protein-IRS1 interaction is required for in vivo tumor growth of mutant cancer cells. Thus, another aspect of the present invention relates to a method of treating a cancer in a subject. The method includes administering to cancer cells in the subject a therapeutically effective amount of an agent that inhibits binding of a p110α mutant protein to IRS1 in the cells, thereby inhibiting the catalytic activity, signaling, and function of the p110α mutant protein in the cell.

The cancer cells to be targeted for therapeutic administration of the agents and compositions described herein will be cancer cells expressing a p110α mutant protein (e.g., a p110α E545K mutant helical domain protein). The cancer cells targeted for therapeutic administration of the agents and compositions described herein may be located in a tumor site (i.e., tumor cells). The cancer cells targeted for therapeutic administration of the agents and compositions described herein can include a solid tumor cell, such as a lung cancer cell, a brain cancer cell, a head & neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a skin cancer cell or a esophageal cancer cell. In certain embodiments, cancer cells targeted for therapeutic administration of the agents and compositions described herein can include carcinoma cells such as colorectal, large cell cancer of the lung and breast carcinoma cells expressing a p110α mutant protein.

In one embodiment, the administration is specific for one or more particular locations within the subject (e.g., a tumor site). The preferred mode of administration can vary depending upon the particular agent chosen and the particular site of the cancer cells or tumor.

A therapeutically effective amount is typically determined in appropriately designed clinical trials (dose range studies) and the person versed in the art will know how to properly conduct such trials in order to determine the effective amount. As generally known, an effective amount depends on a variety of factors including the affinity of the therapeutic agent to IRS1, its distribution profile within the body, a variety of pharmacological parameters such as half life in the body, on undesired side effects, if any, on factors such as age and gender, etc. The therapeutically effective amount of a therapeutic agent may be varied or adjusted widely depending upon the particular application, the manner or introduction, the potency of the particular compound, and the desired concentration. In some embodiments, the therapeutically effective amount may be the amount required to inhibit growth, proliferation, survival, and/or motility of the cancer or tumor cells in the subject.

In some embodiments, a test sample can be taken from the subject in order to determine if the subject's tumor or cancer cells express oncogenic p110α mutant protein associated with cancer or if the cells include one or more genetic mutations identified in a PIK3CA coding sequence where the genetic mutation encodes a mutant p110 protein that directly interacts with IRS1 independently of the p85 regulatory subunit of PI3Kα. Once such a mutation has been identified in the test tissue, the subject may be deemed as a good candidate for therapy with an agent of the present invention.

Where clinical applications are contemplated, it will be necessary to prepare therapeutic agents in pharmaceutical compositions in a form appropriate for the intended application. Therefore, another aspect of the present invention relates to a pharmaceutical composition including a therapeutic agent described herein and a pharmaceutically acceptable carrier. The therapeutic agent inhibiting binding of a p110α mutant protein to IRS1 when administered to a cancer cell expressing the p110α mutant. Generally, this will entail preparing compositions that are essentially free of pyrogens, as well as other impurities that could be harmful to humans or animals.

The phrases “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the therapeutic agent, its use in the pharmaceutical compositions is contemplated. Supplementary active ingredients can also be incorporated into the compositions.

When the pharmaceutical compositions are delivered to a subject, they can be administered by any suitable route, including, for example, orally (e.g., in capsules, suspensions or tablets) or by parenteral administration. Parenteral administration can include, for example, intratumoral, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally.

Administration can be local or systemic as indicated. Desirable features of local administration include achieving effective local concentrations of the therapeutic agent as well as avoiding adverse side effects from systemic administration of the therapeutic agent. In one embodiment, the therapeutic agent can be administered by directly injecting a therapeutic agent into a tumor site, i.e., intratumoral administration. Such compositions would normally be administered as pharmaceutically acceptable compositions, described supra.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

The pharmaceutically acceptable compositions can be suspended in aqueous vehicles and introduced through conventional hypodermic needles or using infusion pumps. For injection, pharmaceutical compositions can be formulated in liquid solutions, typically in physiologically compatible buffers such as Hank's solution or Ringer's solution. In addition, the therapeutic agent may be formulated in solid form and re-dissolved or suspended immediately prior to use. Lyophilized forms are also included. The injection can be, for example, in the form of a bolus injection or continuous infusion (such as using infusion pumps) of the therapeutic agent.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions described herein may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In some embodiments, the therapeutic agent can administered to a subject for an extended period of time to produce optimum inhibition of cancer cell growth, proliferation, survival and/or motility. Sustained contact with the active compound can be achieved, for example, by repeated administration of the active compound(s) over a period of time, such as one week, several weeks, one month or longer (either by repeated administration or by use of a sustained delivery system, or both). In one particular embodiment, the therapeutic agent can be administered to the subject by directly injecting the agent into a tumor site daily for 14 days.

Sustained delivery of the therapeutic agent can be demonstrated by, for example, the continued therapeutic effect of the therapeutic agent over time (such as sustained delivery of the agents can be demonstrated by continued inhibition of cancer or tumor cell growth in a subject). Alternatively, sustained delivery of the therapeutic agent may be demonstrated by detecting the presence of the therapeutic agents in vivo over time.

It is common in many fields of medicine to treat a disease with multiple therapeutic modalities, often called “combination therapies.” To treat cancers associated with the activity, signaling and/or function of p110α mutant proteins using the methods and compositions of the present invention, one would generally administer a therapeutic agent described herein to a target cell or subject and at least one other therapy. These therapies would be provided in a combined amount effective to achieve a reduction in one or more disease parameter. This process may involve administering both agents/therapies to the cells/subjects at the same time, e.g., using a single composition or pharmacological formulation that includes both agents, or by administering to the cell/subject two distinct compositions or formulations, at the same time, wherein one composition includes a therapeutic agent described herein capable of inhibiting binding of a p110α mutant protein to IRS1 in the cancer cell of a subject and the other includes the other agent.

Alternatively, the therapeutic agent of the present invention may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would administer to the cell both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the therapeutic agent or compositions of the present invention or the other therapy will be desired. Other combinations are contemplated.

In the instances of combination therapies described herein, it will be understood the administration further includes a pharmaceutically or therapeutically effective amount of the additional therapeutic agent in question. The second or additional therapeutic agents described herein may be administered in the doses and regimens known in the art or may be administered in low doses.

Agents or factors suitable for use in a combined therapy may include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” are intended to be of use in the combined treatment methods disclosed herein.

In some embodiments, the other therapeutic agent used in a combination therapy can include a p110α inhibitor selected from LY294002, wortmannin and derivatives of LY294002 and wortmannin with more favorable pharmacological profiles.

In some embodiments, the other therapeutic agent used in a combination therapy can include at least one anti-proliferative agent. The phrase “anti-proliferative agent” can include agents that exert antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development, which could be included in the present invention by combination drug chemotherapy. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloid.

Other therapies that cause DNA damage and have been used extensively include what are commonly known as γ-rays, x-rays, and/or the directed delivery of radioisotopes to cancer and tumor cells. Other forms of DNA damaging factors are also contemplated such as microwaves and UV irradiation. It is most likely that all of these factors affect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition to combining therapeutic agent therapies described herein with chemo- and radiotherapies, it also is contemplated that combination with immunotherapy, hormone therapy, toxin therapy and surgery. In particular, one may employ targeted therapies such as Avastin, Erbitux, Gleevec, Herceptin and Rituxan.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example

This Example shows that disruption of the interactions between helical domain p110α mutants and IRS1 may be exploited as a more accessible targeted therapy approach for patients having cancers harboring such mutations. Further, given that the mutant p110α-IRS1 interaction only exists in tumors harboring these mutations, it is likely that drugs targeting this interaction should have no or minimal toxicity.

The current paradigm is that the helical domain mutations weaken p110α's interactions with the p85 regulatory subunits, alleviate the inhibitory effect of p85, and thus increase the enzymatic activity of p110α. In contrast, our results describe below suggest that the weakened p110α-p85 interaction caused by the mutations is not sufficient for the p110α-helical domain mutant proteins to exert their oncogenic functions. We show several pieces of evidence that the p110α E545K-IRS1 interaction plays a critical role in tumorigenesis. Mechanistically, our data suggest that p110α E545K mutant-IRS1 interaction activates the mutant p110α lipid kinase by facilitating the localization of the mutant enzyme complexes from cytosol to plasma membrane and by stabilizing the mutant p110α proteins. We show that the helical domain mutations of p110α, as well as some of the mutations in the ABD and C2 domains, induce conformational changes that cause p110α to acquire a direct protein interaction with IRS1, which does not require IRS1 tyrosine phosphorylation and p85 proteins.

### Methods

### Tissue Culture

Colorectal cancer (CRC) cell lines DLD1, HCT116, Vaco481 and genetically engineered CRC cell lines were maintained in McCoy's 5A medium containing 10% fetal bovine serum (FBS). Lung cancer cell line H460 and breast cancer cell line T47D were cultured in RPMI 1640 medium plus 10% of FBS. Breast cancer cell line MDA-MB361 was maintained in Leibovitz's L-15 medium with 20% FBS. Human embryonic kidney HEK293 cells were cultured in DMEM medium containing 10% FBS. Mammalian cells were transfected with Lipofectamine in accordance with the manufacturer's instructions (Invitrogen).

### Somatic Gene Targeting

The targeting vectors were constructed with the USER system and targeted cells were generated as described previously (Du et al., 2010). Briefly, vector arms were created by PCR from normal human DNA using HiFi Taq (Invitrogen) and validated by sequencing prior to virus production and infection. The parental DLD1 cells were purchased from ATCC (American Type Culture Collection) and infected with indicated rAAV viruses. Stable G418-resistant clones were then selected for PCR screening as reported. When relevant, targeted clones were genotyped by RT-PCR and sequencing when necessary. Primers for targeting vector construction and PCR screening are listed in the Supplemental Experimental Procedures.

### Immunoprecipitation and Immunoblotting

For Co-immunoprecipitation (Co-IP), cells were harvested, washed with PBS, and then lysed in IP buffer (50 mMTris [pH 7.4], 150 mMNaCl, 5 mMEDTA [pH 8.0], 0.5% NP40, 1 mM PMSF, complete Protease Inhibitor Cocktail tablet [Roche]; supplemented with phosphatase inhibitors [1 mM Na3VO4, 20 mM NaF, 0.1 mM b-glycerophosphate, 20 mM sodium pyrophosphate] when necessary). For immunoblotting (IB) of phospho-specific proteins, unless stated otherwise, cells were lysed in urea buffer (100 mM NaH₂PO₄, 10 mM Tris-HCl, 8 M Urea, 1 mM Na₃VO₄, 20 mM NaF, 0.1 mM β-glycerophosphate, 20 mM sodium pyrophosphate, supplemented with complete Protease Inhibitor Cocktail tablet [Roche], pH 8.0). Lysates were then cleared by centrifugation (14,000 rpm, 10 min) and protein concentration in supernatants was determined with a BCA protein assay kit (Pierce, Rockford, Ill.). Equal amounts of total protein were used for IP or IB.

### In Vitro Binding Assay

The 6×His-MYC-IRS1 baculoviral vector was constructed using the bac-tobac expression system (Invitrogen). The 6 3 His-p110 α WT and 6 3 Hisp110α E545K baculoviral vectors were kind gifts from Dr. Bert Vogelstein. These recombinant proteins were expressed in Sf9 cells and purified using Ni-NTA agarose beads as described (Yu et al., 2008). For in vitro binding, 100 ng of purified 6 3 His-MYC-IRS1 proteins were first attached to anti-Myc antibody-bound beads and then incubated with the purified 6 3 Hisp110 α WT or 6 3 His-p110 α E545K proteins in 1 ml of IP buffer. The protein-bound beads were washed three times for western blot analysis.

### Pull-Down Assay

His-tagged or GST-tagged truncated IRS1 plasmids were constructed with PCR-based subcloning using USER system. Each construct was transformed into E. coli strain BL21. Recombinant protein expression was induced with 0.2 mM IPTG at 16° C. overnight. His-tagged and GST-fusion proteins were purified using either Ni-NTA agarose beads or Glutathione Sepharose 4B beads according to manufacturer's instructions. The purified recombinant proteins were incubated with cell lysates prepared as described previously.

### Peptide Competition Assay

For in vitro peptide competition assay, the p110α mutant-FLAG-tagged cells were grown to 90% confluency, serum-starved overnight, and lysed as described previously. Peptides were then added to the cell lysates, incubated for 30 min at 4° C. and followed by immunoprecipitation assay.

### Xenografts

Animal experiments were approved by the Case Western Reserve University Animal Care and Use Committee. 3 million cells were injected subcutaneously into the flanks of 4- to 6-week-old female athymic nude mice. Tumor volume was measured with electronic calipers, and volumes were calculated as length 3 width²/2.

### Peptide Treatment of Xenograft Tumor

Five-week-old male athymic nude mice were injected with 5 million cells subcutaneously and bilaterally. When tumor sizes reached 100-150 mm³, 10 mice with similar sized tumors on both flanks were selected. The left flank tumors were injected with stapled WT p110α peptide, whereas the right flank tumors were injected with stapled p110α E545K mutant peptide. Each tumor was injected with 250 μg of peptide daily for 14 days. Control xenograft tumors were injected with an equal volume of water. The tumor sizes were measured every 2 days.

### Plasmid Construction

The cDNAs of p110α, IRS1, p85α were purchased from Addgene. Plasmids of truncated IRS1 were constructed by PCR-based subcloning into various vectors using USER system (New England Biolabs). Site-directed mutagenesis was carried out with Quickchange Kit (Stratagen).

### Subcellular Fractionation

Twenty million cells were scraped from plates in Subcellular Fractionation Buffer (SFB) [250 mM Sucrose, 20 mM HEPES, 10 mM KCl, 1.5 mM MgCl₂, 1 mM EDTA, 1 mM EGTA, 1 mM DTT and Protease inhibitor cocktail], and collected in 1.5 ml eppendorf tube. Cell lysates were passed through a 27½ G needle 10 times using a 1 ml syringe, sit on ice for 20 minutes and centrifuged at 8000 rpm to remove cell debris, nuclear and mitochondria et al. in the pellets. Supernatants were transferred into a fresh tube and centrifuged at 40000 rpm for 1 hour. The supernatants were saved as the cytoplasmic fractionation. Pellets were then washed, resuspended in SFB buffer and passed through a needle as described above. The lysates were re-centrifuged at 40000 rpm for 45 minutes. The pellets were dissolved in 200 μl of lysis buffer as the membrane fractionation. Western blot analyses were used to quantify proteins in each fraction. The p110α protein was normalization against controls. The ratios of membrane faction were calculated according to the following formula:

Amount of membrane faction/(Amount of cytoplasmic faction×dilution factor+Amount of membrane faction).

### In Vitro Lipid Kinase Assay

p110α E545K mutant and p85 niSH2 proteins were co-expressed in sf9 cells as described before (Huang et al., 2007). The mutant p110α proteins were purified using Ni-NTA beads. The purified p110α proteins were used for in vitro lipid kinase assay as described (Samuels et al., 2005). Briefly, 10 nM of p110α E545K mutant protein were added into each reaction containing 1 mg/ml Phosphatidylinositol (Sigma, St. Louis, Mo.), 10 mM Hepes, 2.5 mM MgCl₂, 25 μM ATP and 30 μCi of [γ-³²P] ATP. The reactions were incubated at room temperature for 10 minutes, and then stopped by adding 75 μl of 1 M HCl. Lipids were subsequently extracted using 150 μl of chloroform:methanol (1:1) (v:v) and then washed once suing 1 M HCl:methanol (1:1) (v:v). The organic phase was loaded onto pretreated and activated Silica Gel 60 TLC plates. The plates were developed in chloroform:methanol: 4 M NH4OH (9:7:2) (v: v) and visualized by autoradiography.

### Peptide Synthesis

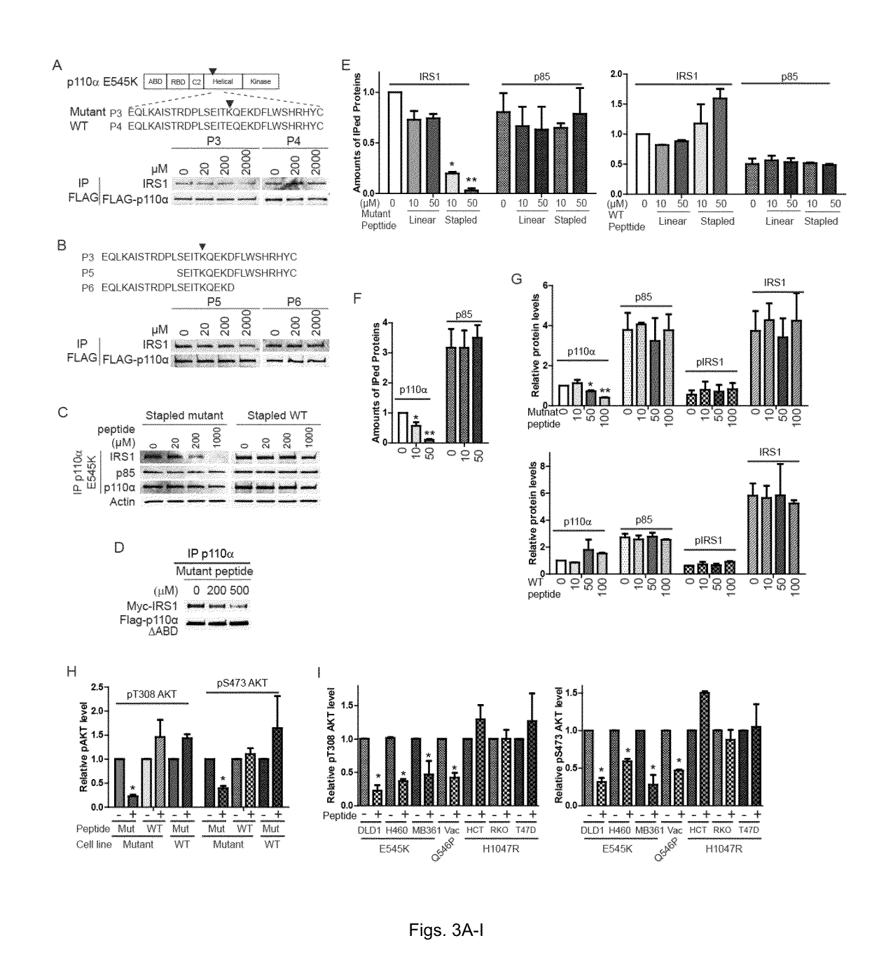

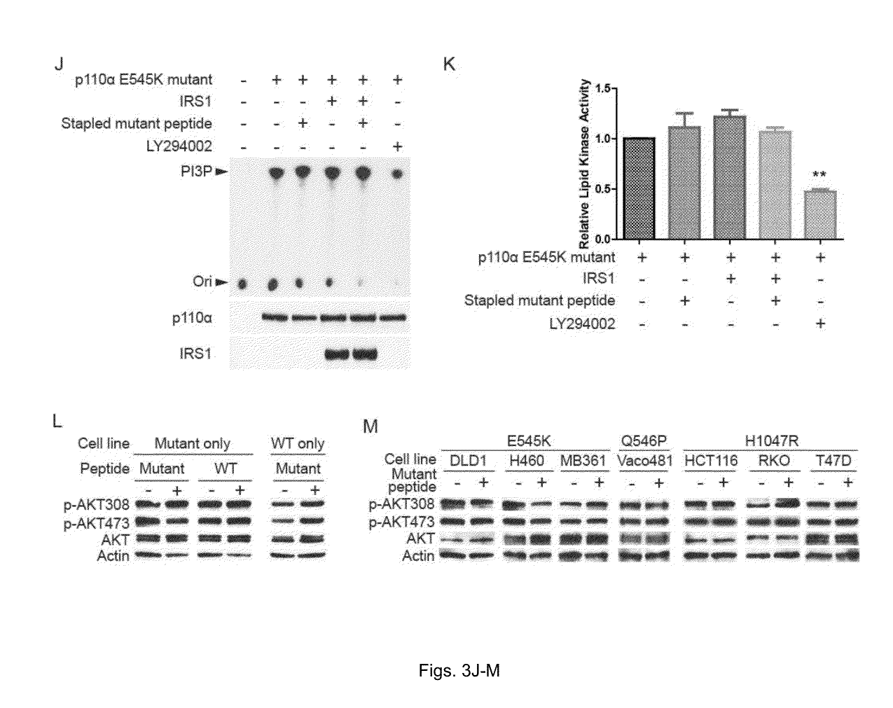

The 9-fluorenylmethoxycarbonyl (Fmoc) chemistry-based solid phase peptide synthesis (SPPS) was performed on a PS3 peptide synthesizer (Protein Technologies Inc., Tucson, Ariz., USA). All the linear peptides shown in FIG. 4, and FIG. 3 were synthesized on pre-loaded Wang or 2-chlorotrityl resins (Novabiochem, La Jolla, Calif., USA), and the two stapled peptides shown in FIG. 4 were synthesized on the Rink Amide MBHA resin (Novabiochem). All the Fmoc-protected amino acids were purchased from Novabiochem except (S)-N-Fmoc-2-(4′-pentenyl) alanine which was purchased from AnaSpec (Fremont, Calif., USA). During the synthesis of the two stapled peptides, this latter amino acid building block was used to replace both D549 and 5553 in H₂N-SEITKQEK-D⁵⁴⁹-FLW-S⁵⁵³-HRHYC-COOH (SEQ ID NO: 8) and H₂N-SEITEQEKD⁵⁴⁹-FLW-S⁵⁵³-HRHYC-COOH (SEQ ID NO: 7). It should be pointed out that, the introduction of the 4′-pentenyl side chain at these two positions for the subsequent on-resin ring-closing olefin metathesis could potentially furnish a stabilized α-helix since they are the corresponding i and i+4 positions of an α-helix, which is a well-utilized side chain relative positioning for peptide stapling. Furthermore, based on the crystal structure of p110α (Huang et al., 2007,) the macrocyclic bridging unit of the resulting stabilized α-helix would incur a minimum steric clash with the neighboring structural motifs for binding to IRS1.

For each coupling reaction during the assembly of the peptidyl chain on the peptide synthesizer, 4 equivalents of a Fmoc-protected amino acid (or 2-3 equivalents of (S)-N-Fmoc-2-(4′-pentenyl)alanine), 3.8 equivalents of the coupling reagent 2-(1H-Benzotriazole-1-yl)-1,1,3,3-tetramethyluronium hexafluorophosphate (HBTU) (or 1.9-2.85 equivalents of the coupling reagent 2-(7-Aza-1H-benzotriazole-1-yl)-1,1,3,3-tetramethyluronium hexafluorophosphate (HATU) for (S)-N-Fmoc-2-(4′-pentenyl)alanine) and the additive N-hydroxybenzotriazole (HOBt, for the HBTU-mediated coupling only) were used in the presence of 0.4 M 4-methylmorpholine (NMM)/DMF, and the coupling reaction was allowed to proceed at room temperature for 1 h (9 h or 2 h for (S)-N-Fmoc-2-(4′-pentenyl)alanine). A 20% (v/v) solution of piperidine in N,N-Dimethylformamide (DMF) was used for Fmoc removal. Of note, HATU was used as the coupling reagent for the double coupling of the amino acid immediately N-terminal to (S)-N-Fmoc-2-(4′-pentenyl)alanine, i.e., K and W.

For the synthesis of the linear peptides P1-P6, the N-terminal Fmoc group of the peptidyl resin was removed with the 20% (v/v) piperidine/DMF solution before a fully unprotected peptide was cleaved from the resin at room temperature for 4 h by a trifluoroacetic acid (TFA)-containing cleavage cocktail (83.6% (v/v) TFA, 5.9% (v/v) phenol, 4.2% (v/v) ddH₂O, 4.2% (v/v) thioanisole, 2.1% (v/v) ethanedithiol), precipitated in cold diethyl ether, and dried by lyophilization. The purification of the resulting crude peptide was performed with the reversed-phase HPLC on a semi-preparative Microsorb C18 column (100 Å, 5 μm, 250×10 mm). The column was eluted with a gradient of ddH₂O containing 0.05% (v/v) of TFA and acetonitrile containing 0.05% (v/v) of TFA at 5 mL/min and monitored at 214 nm. The pooled HPLC fractions were stripped of acetonitrile and lyophilized to afford an isolated peptide as a puffy white solid. The exact masses of P3-P6 were confirmed by matrix assisted laser desorption ionization-time of flight (MALDI-TOF) mass spectrometric analysis. P3: Calcd. for [M+H]⁺3658.8; found: 3660.3. P4: Calcd. for [M+H]+3659.8; found: 3660.4. P5: Calcd. for [M+H]⁺2307.1; found: 2307.9. P6: Calcd. for [M+H]+2429.3; found: 2430.1.

For the synthesis of the two stapled peptides, the N-terminal Fmoc group of a peptidyl resin was removed with the 20% (v/v) piperidine/DMF solution before the exposed α amino group was acetylated with neat acetic anhydride at room temperature for 1 h. The resulting peptidyl resin was harvested and washed with DMF and dichloromethane (DCM), and dried in vacuum before it was treated with a 10 mM solution of Bis(tricyclohexylphosphine)benzylidine ruthenium(IV) dichloride (Grubbs catalyst, 1st generation, Aldrich) in the freshly degassed (with N₂ gas) 1,2-dichloroethane (4 mL for a 0.1 mmole scale of the peptide synthesis) for 2×2 h at room temperature. The resulting dark brown peptidyl resin was subsequently washed with 1,2-dichloroethane and DCM before the side chain fully unprotected stapled peptide was cleaved from the resin at room temperature for 4 h by the above-described TFA-containing cleavage cocktail, precipitated in cold diethyl ether, and dried by lyophilization. The purification of a crude stapled peptide was also performed with the reversed-phase HPLC on the above-described semi-preparative C18 column, and followed the same procedure as that described above for the purification of a linear peptide. After the lyophilization of the pooled HPLC fractions, a stapled peptide was isolated as a puffy white solid. The exact masses of the two stapled peptides were also confirmed by MALDI-TOF mass spectrometric analysis. Mutant stapled peptide: Calcd. for [M+H]⁺2396.2; found: 2396.3. WT stapled peptide: Calcd. for [M+H]⁺2397.6; found: 2397.4.

### Results

The p110α E545K Mutant, but not the Wild-Type p110α, Associates with IRS1

DLD1 is a CRC cell line that carries a wild-type (WT) PIK3CA allele and a PIK3CA E545K allele. DLD1 derivatives carry only the endogenous WT or the mutant PIK3CA allele that had been generated previously. To identify proteins that may differentially bind to WT and E545K mutant p110α, we used recombinant adeno-associated virus (rAAV)-mediated homologous recombination to tag the endogenous WT or mutant p110α with 3×FLAG at their C termini in DLD1 cells. Under serum-starvation conditions, antibodies against FLAG immunoprecipitated a protein of ˜170 kDa from 3×FLAG-tagged p110α E545K knock-in (KI) cells, but not from the 3×FLAG-WT p110α KI cells. Analysis by mass spectrometry identified this protein as IRS1. This IRS1-p110α E545K interaction was validated by immunoprecipitation under serum-starvation conditions in three different settings. In KI DLD1 cells, IRS1 was co-immunoprecipitated with p110α E545K but not the WT p110α. Moreover, when IRS1 was immunoprecipitated from various DLD1 derivatives, p110α E545K predominantly associated with IRS1. Furthermore, IRS1 is strongly associated with mutant p110α in the mutant only DLD1 cells but its interaction with the WT p110α was barely detectable in the WT-only cells.

p110α-Helical Domain Mutants, but not the Kinase Domain Mutants, Interact with IRS1

In addition to the hot-spot mutations in the helical and kinase domains, cancer-derived mutations also occur in the ABD and C2 domains. We thus proceeded to test whether gain of interaction with IRS1 occurs with other p110α mutations. We constructed a FLAG-tagged p110α expression plasmid and generated frequently observed tumor-derived p110α mutations by site-directed mutagenesis. The p110α WT or mutant plasmids were coexpressed with a MYC-tagged IRS1 construct in HEK293 cells for immunoprecipitations. In addition to the E545K mutation, other hot-spot p110α mutations in the helical domain, including the E542K, E545A, E545G, and Q546K, gain interaction with IRS1 under serum-starvation conditions. In contrast, however, hot-spot mutations in the kinase domain, H1047R, H1047L, and G1049R, failed to interact with IRS1. Interestingly, relatively rare mutations including K111N in the ABD domain, N345K in the C2 domain, and M1043L in the kinase domain also interact with IRS1, whereas other rare mutations including R88Q in the ABD domain, C420R mutations in the C2 domain and P539R in the helical domain, failed to interact with IRS1.

Interaction Between the Helical Domain Mutant p110α and IRS1 does not Require p85 or IRS1 Phosphorylation

Given that p110α is normally brought to the IRS1 complex through the interaction between the SH2 domain of p85 and phosphorylated tyrosine (pY) residues of IRS1, it was important to determine whether the p110α E545K-IRS1 interaction required p85 and tyrosine phosphorylation of IRS1. p110α E545K, but not the WT p110α, robustly associated with IRS1 under serum-starvation conditions in which tyrosine phosphorylation of IRS1 was undetectable, suggesting that the p110α E545K-IRS1 interaction is independent of IRS1 tyrosine phosphorylation. It appeared that the p110α E545K-IRS1 interaction also did not require p85 because a truncated p110α E545K lacking the ABD domain required for p85 binding. We then proceeded to determine whether p85 proteins were required for the interaction between p110α E545K and IRS1 under physiologic conditions. This was accomplished by knocking out PIK3R2 (encodes p850β) in the p110α mutant only DLD1 cells using rAAV-mediated homologous recombination and knocking down p85α with a siRNA in the p85β knockout (KO) cells. Ablation of p85α and p85β enhanced rather than inhibited the interaction between p110α E545K and IRS1, suggesting that not only p85 proteins were not required, but also may compete with IRS1 for binding to p110α E545K. However, ablation of both p85 proteins led to destabilization of p110α protein. Together, our data suggest that p85 proteins are not required for the interaction between p110α E545K and IRS1 but still play a role in the protein complex through stabilization of the mutant p110α. Consistently, p85 proteins were coimmunoprecipitated with p110α E545K. Furthermore, gel filtration analysis showed that p85 and p110α E545K formed a protein complex that was larger than that formed by p85 and the WT p110α. Interestingly, p110α E545K completely cofractionated with IRS1, while only a portion of p85 proteins cofractionated with p110α E545K and IRS1. This result shows that the p110α E545K mutation changes protein complex formation.

To test if IRS1 directly binds to p110α E545K, we expressed recombinant IRS1, WT p110α, and p110α E545K in Sf9 cells and purified these proteins to homogeneity. Recombinant IRS1 bound directly in vitro with p110α E545K but not WT p110α, providing further evidence that the IRS1-p110α E545K interaction is not mediated by p85 proteins.

Specific Regions of IRS1 are Required for its Interaction with p110α E545K

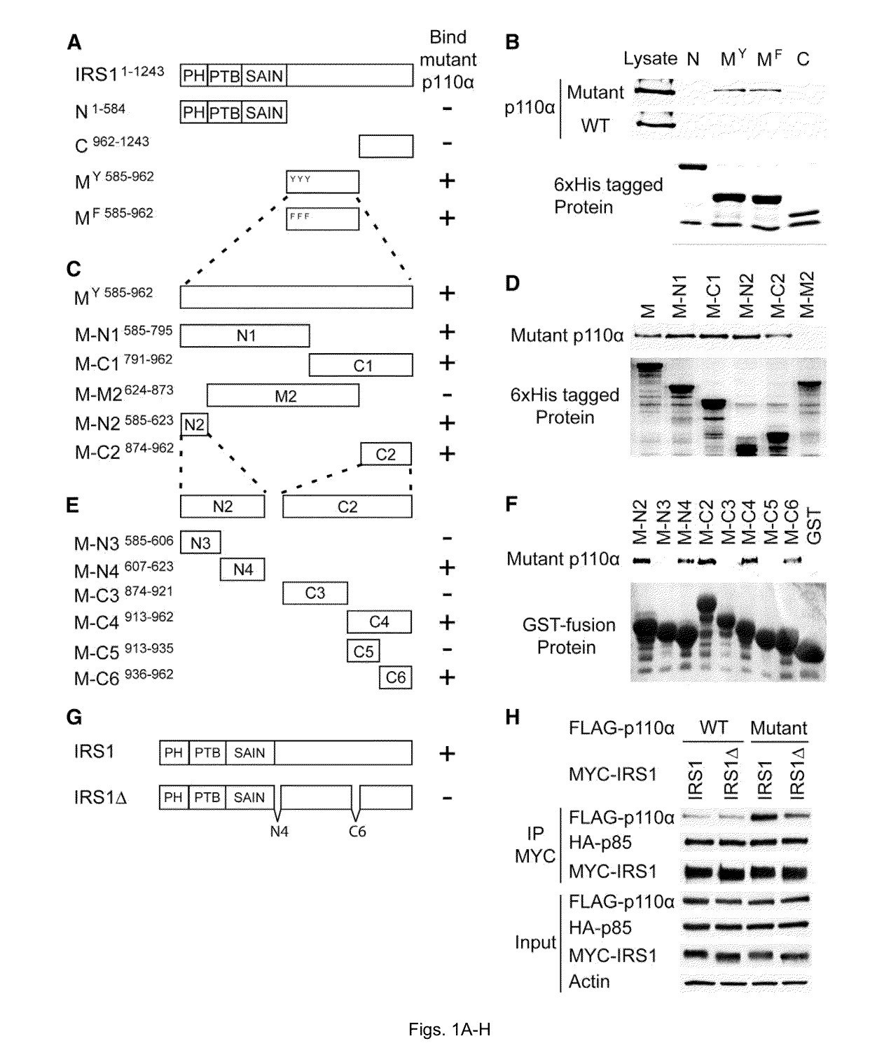

The altered interaction properties of the mutant forms of p110α prompted us to map the minimal essential regions of IRS1 required for its interaction with p110α E545K mutant protein. For this study, we made a series of 6×His-tagged IRS1 truncation constructs (FIGS. 1A and 1C). The His-tagged IRS1 proteins were first expressed in Eschericia coli and purified with Ni-NTA beads. Then the purified proteins were used to pull down p110α from DLD1 cells expressing either the WT p110α only or the p110α E545K mutant only. With these proteins, we first mapped the mutant p110α interaction domain to the middle part of IRS1 (amino acid [AA] 585-962) (FIG. 1B). This fragment contains three YxxM motifs that mediate the pY dependent IRS1-p85 protein interaction. We thus constructed an unphosphorylatable IRS1Y3F mutant fragment (FIG. 1A). As shown in FIG. 1B, this mutant fragment still bound to p110α E545K, providing further evidence that the protein interaction between IRS1 and p110α E545K mutant is independent of IRS1 phosphorylation. Moreover, we showed that the middle fragment of IRS1 pulled down the p110α E545K, but not the WT counterparts (FIG. 1B). Then, we further mapped the interaction domains to two small regions (AA 607-623 and AA 936-962) in the middle part of IRS1 using a combination of 6×His-tag and GST-fusion constructs (FIG. 1C-1F). When expressed in HEK293 cells, a deletion construct of IRS1 (IRS1D) devoid of both AA 607-623 and AA 936-962 regions greatly reduced its interaction with p110α E545K under both normal tissue culture and serum-starvation conditions (FIGS. 1G and 1H). In contrast, neither of the single region deletions could interrupt the IRS1 mutant p110α interaction. Importantly, however, IRS1D did not affect the IRS1-p85 interaction (FIG. 1H), providing further supporting evidence that the IRS1 mutant p110α interaction does not require p85. Finally, similar results were also observed when the IRS1D was expressed in p110α E545K 3×FLAG KI DLD1 cells.

IRS1 Stabilizes p110α

Next, we set out to develop a system to test whether disruption of the IRS1 mutant p110α interaction affects tumorigenicity. To this end, we first knocked out IRS1, a single exon gene, from DLD1 cells (IRS1 KO) using rAAV-mediated homologous recombination. Two independently-derived IRS1 KO clones were then chosen for further analyses. Interestingly, p110α, but not p85, protein levels were reduced in the IRS1 KO cells in comparison to the parental cells, suggesting that IRS1 stabilizes p110α. It appears that the interaction between IRS1 and p110α E545K is required to stabilize p110α E545K, and coexpression of IRS1 with p110α in Sf9 insect cells stabilizes p110α E545K but not the WT p110α.

IRS1-p110α E545K Interaction Brings the Mutant p110α to the Cytoplasmic Membrane

Given that membrane association of p110α is important for its activation, we then determined how IRS1 affected association of the mutant p110α protein with the membrane. Indeed, more p110α E545K bound to cytoplasmic membrane than the WT proteins. Importantly, knockout of IRS1 in the p110α E545K mutant only DLD1 cells reduced the amount of membrane-bound p110α mutant proteins, supporting the conclusion that interaction between IRS1 and p110α E545K enhances the association of p110α E545K with the cytoplasmic membrane.

The p110α E545K-IRS1 Interaction is Required for In Vivo Tumor Growth

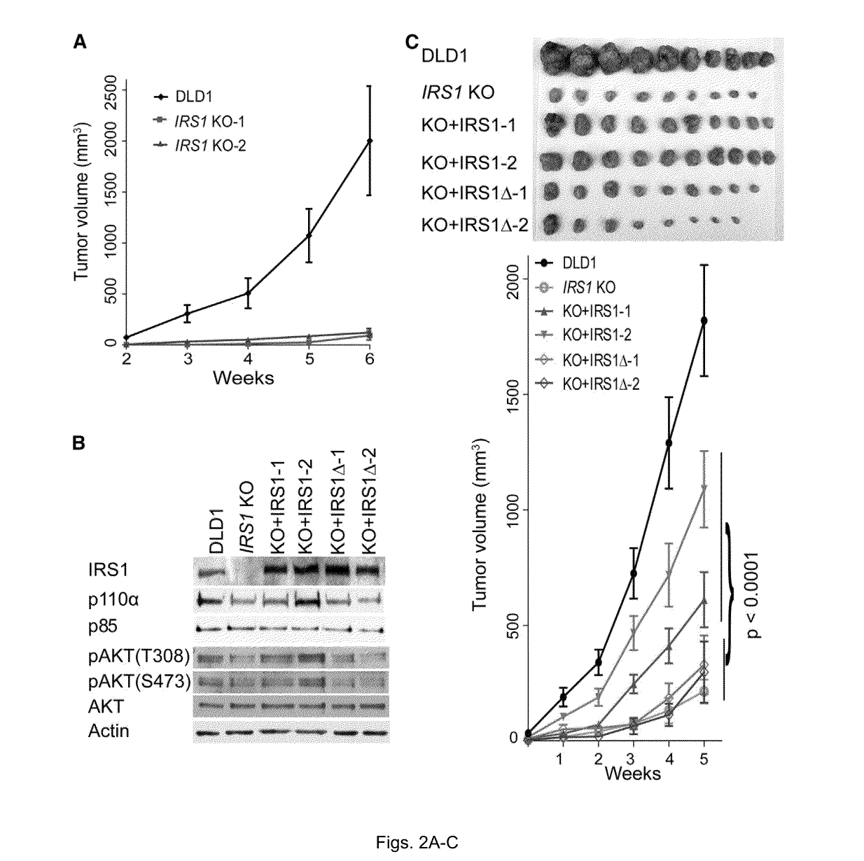

The IRS1 KO cells formed much smaller xenograft tumors in athymic mice (FIG. 2A), although IRS1 KO did not affect clonogenicity and anchorage-independent growth of DLD1 cells under normal tissue culture conditions (data not shown). We reconstituted one of the two IRS1 KO clones with either fulllength Myc-IRS1 or Myc-IRS1D expression plasmid. Two stable clones expressing each construct were chosen for in-depth analyses. Expression levels of IRS1 in the IRS1D reconstituted clones were similar to those of IRS1 reconstituted clones (FIG. 2B). Consistent with the hypothesis that IRS1 stabilizes p110α, IRS1, but not IRS1D, restored the p110α level to one that was comparable to that observed in parental cells (FIG. 2B). Moreover, AKT phosphorylation levels were also increased in IRS1 reconstituted cells in comparison to those of IRS1 KO and IRS1D reconstituted clones under serum starvation (FIG. 2B), suggesting that stabilization of p110α by IRS1 activates downstream signaling. Importantly, both IRS1 reconstituted clones and IRS1D reconstituted clones responded to insulin stimulation, suggesting that IRS1D does not impair cell signaling pathways activated by growth factors. Notably, FIG. 2C shows that IRS1 reconstituted clones formed significantly bigger xenograft tumors than IRS1D reconstituted clones. Consistently, the IRS1-p110α interaction was also impaired in the xenograft tumors formed by the IRS1D reconstituted cells, which led to reduced phosphorylation of AKT and Foxo1 proteins. These results suggest that IRS1-p110α E545K interaction is crucial for the mutant p110α to exert its oncogenic functions. Unexpectedly, the full-length IRS1 reconstituted cells formed smaller tumors than the parental cells. We postulate that this difference could be caused by differences in regulating IRS1 between the parental cells and the IRS1 reconstituted cells. The ectopically expressed IRS1 in the IRS1 reconstituted cells may not recapitulate the complex regulation of the endogenous IRS1. In addition, the ectopically expressed IRS1 protein has a 3xMyc tag at the N terminus of the protein, which may not provide full-function equivalency to the endogenous IRS1 proteins.

A Peptide Derived from p110α E545K Disrupts the p110α-Helical Domain Mutant-IRS1 Interaction

Because our results indicated that the p110α E545K-IRS1 interaction is required for in vivo tumor growth of the mutant CRC cells, we set out to test whether p110α-derived peptides encompassing the mutation site could be constructed to disrupt this interaction. Based on the crystal structure of p110α, we first synthesized a 30-AA E545K mutant peptide and its WT counterpart (FIG. 3A). These amino acids form two a-helix domains flanking the mutation site. As shown in FIG. 3A, the 30-AA mutant peptide inhibited the interaction between p110α E545K and IRS1 when added into cell lysates, whereas the WT peptide failed to cause any inhibition. We then tested if peptides corresponding to only one of the two a-helical motifs could inhibit interactions between the mutant p110α and IRS1. These experiments demonstrated that an 18-AA mutant peptide corresponding to the C-terminal a-helical motif displayed moderate inhibition in cell lysates (FIG. 3B), while the N-terminal 21-AA mutant peptide had no effect (FIG. 3B). To improve the potency of the peptide, we converted the aforementioned 18-AA mutant peptide to a stapled macrocyclic derivative as shown in FIG. 4A. This approach exploited the well-documented concept that peptide stapling can stabilize the a-helical conformation of a linear peptide and potentially enhance binding affinity toward specific targets. Moreover, this approach was taken because stapled peptides are often cell-permeable. Strikingly, our stapled mutant peptide almost completely disrupted the IRS1-p110α E545K interaction in cell lysates, but had no effects on p110α-p85 interaction (FIG. 3C). In contrast, the corresponding stapled WT p110α peptide did not inhibit IRS1-p110α E545K interactions (FIG. 3C). Interestingly, as shown in FIG. 3D, the stapled mutant peptide also inhibited the interaction between IRS1 and DABD p110α E545K. Thus this result provided further evidence that the protein interaction between IRS1 and p110α E545K mutant is not mediated by p85.

Importantly, when added to cell culture medium, the stapled mutant peptide effectively disrupted IRS1-p110α E545K interaction in DLD1 cells at 50 mM concentration (FIG. 4B; FIG. 3E), demonstrating both targeting activity and cell permeability of this stapled peptide. Furthermore, neither the linear mutant peptide nor the stapled WT peptide affected the interaction between p110α E545K and IRS1 when added to cultured DLD1 cells (FIG. 4B), providing strong support of the specificity and efficacy of the stapled mutant peptide. Moreover, the p110α E545K stapled peptide also disrupted protein interaction between p110α Q546P and IRS1 in Vaco481 CRC cells (FIG. 4C; FIG. 3F), suggesting that these helical domain mutant proteins share a common critical protein interaction interface with IRS1. In support of earlier results, it was also notable that the stapled mutant peptide did slightly reduce the protein levels of p110α, but did not affect levels of p85 and IRS1 (FIG. 4D; FIG. 3G), providing further evidence that the protein interaction between mutant p110α and IRS1 stabilizes p110α. Finally, consistent with the in vitro results, the stapled mutant peptide did not interfere with cellular p110α-p85 interaction or IRS1 phosphorylation (FIGS. 4B and 4D).

The p110α E545K Mutant Peptide Reduces AKT Phosphorylation in Cancer Cell Lines with p110α-Helical Domain Mutations

We next determined the effect of the stapled peptides on the downstream signaling of PI3K. As shown in FIG. 4E and FIG. 3H, the stapled mutant peptide, but not the WT counterpart, reduced AKT phosphorylation in mutant-only DLD1 cells, whereas the mutant peptide did not affect AKT phosphorylation in WT-only DLD1 cells. Moreover, treatment of cancer cell lines (DLD1, H460, and MDA-MB361) harboring p110α E545K and the Vaco481 cell line harboring p110α Q546P with the stapled mutant peptide resulted in reduced AKT phosphorylation at both T308 and 5473 residues (FIG. 4F; FIG. 3I). Again, in contrast, the stapled mutant peptide had no effect on AKT phosphorylation in cancer cell lines (HCT116, RKO, and T47D) harboring p110α H1047R (FIG. 4F). Importantly, the stapled mutant peptide has no effect on the kinase Cancer Cell p110α Mutant Proteins Rewire Oncogenic Signaling 588 Cancer activity of recombinant p110α, either alone or in the presence of IRS1, in vitro (FIGS. 3J and 3K). Consistent with previous reports that phospho-IRS1 peptide does not enhance the lipid kinase activity of p110α E545K, full-length IRS1 proteins also had no effect on the lipid kinase activity of p110α E545K in vitro (FIGS. 3J and 3K). Thus, the ability of stapled mutant peptide to reduce AKT phosphorylation in cancer cells harboring p110α E545K is not due to direct inhibition of the lipid kinase activity of p110α but by disrupting the interaction between the p110α mutant and IRS1. Consistent with the notions that disruption of IRS1-p110α-helical domain mutant protein interaction does not perturb AKT activation induced by growth factors, the mutant stapled peptide had no effect on AKT phosphorylation when these cell lines were stimulated by insulin (FIGS. 3L and 3M).

The Stapled p110α E545K Mutant Peptide Specifically Inhibits In Vivo Tumor Growth of Cancer Cells with the Mutation

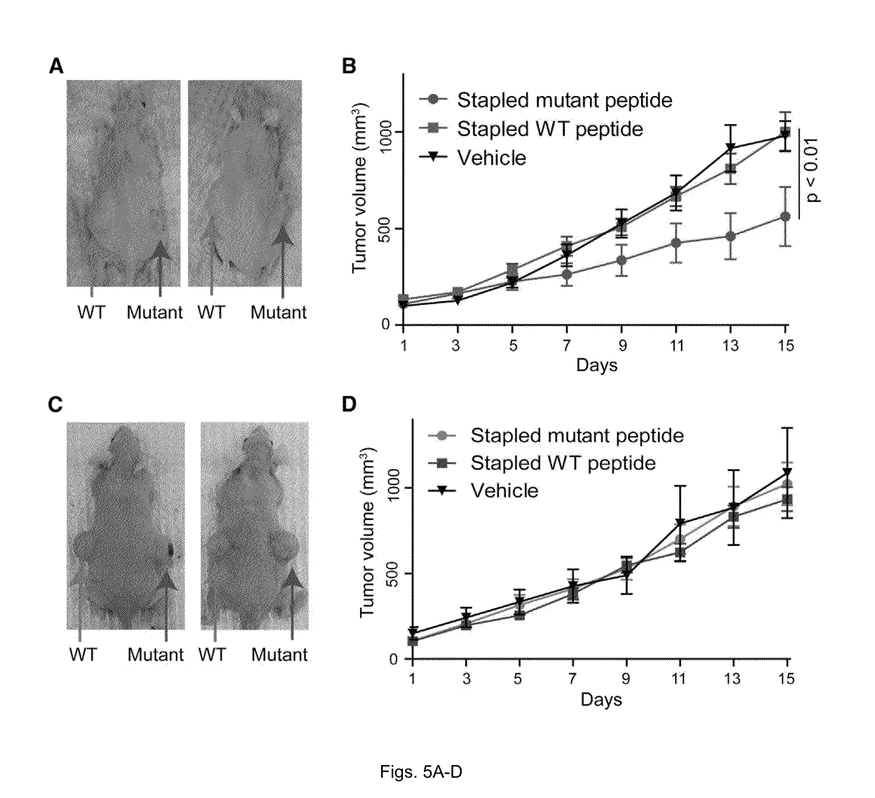

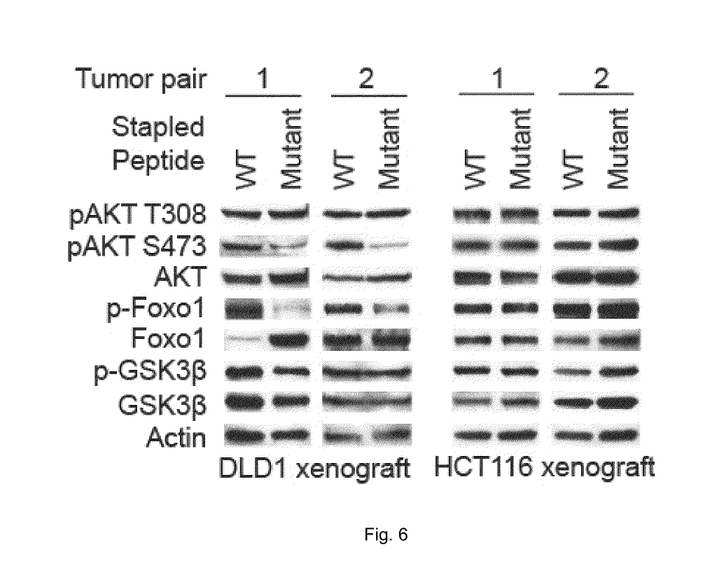

Finally, we tested whether the stapled mutant peptide can inhibit xenograft tumor growth of DLD1 cells. Peptides were directly injected into xenograft tumors when the tumors reached ˜100-150 mm³. To better control the experiment, we injected the stapled WT peptide into tumors on the left flanks and the stapled mutant peptide into tumors on the right flanks of the same nude mice (FIG. 5A). Tumors of similar size were also injected with an equal volume of water as controls. As shown in FIG. 5B, the stapled mutant peptide treatment significantly slowed DLD1 xenograft tumor growth in comparison with the control group. In contrast, the stapled WT peptide did not inhibit xenograft tumor growth of DLD1 cells. Moreover, neither the stapled WT nor the stapled mutant peptide inhibited xenograft tumor growth of HCT116 CRC cells that harbor p110α H1047R (FIGS. 5C and 5D). In the xenograft tumors, the stapled mutant peptide treatment led to reduced AKT and Foxo1 phosphorylation (FIG. 6). Unlike in culture cells where treatment of the stapled mutant peptide reduced AKT phosphorylation at both T308 and S473 residues (FIGS. 4E and 4F), peptide treatment in xenograft tumors only decreased AKT phosphorylation at the S473 residue (FIG. 6), suggesting that the microenvironment in xenograft tumors is more complex than that in in vitro culture conditions. Consistent with our observation, several studies have indicated that phosphorylation of AKT T308 and S473 are regulated differentially.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

Having described the invention, we claim:

1. A method of inhibiting the activity, signaling, and/or function of a p110α mutant protein in a cancer cell expressing the p110α mutant protein, the method comprising administering to the cancer cell an amount of a therapeutic agent effective to inhibit binding of the p110α mutant protein to IRS1 in the cell, the therapeutic agent comprising a polypeptide consisting of about 10 to about 40 amino acids, the polypeptide having at 80% sequence identity with consecutive amino acids of a portion of a helical domain of the p110α mutant protein that includes the mutated amino acid.

2. The method of claim 1, wherein inhibition of binding of the p110α mutant protein to IRS1 thereby inhibits the catalytic activity, signaling, and function of the p110α mutant protein in the cancer cell.

3. The method of claim 1, the amount of the therapeutic agent being an amount effective to inhibit proliferation, survival and/or motility of the cancer cell.

4. The method of claim 1, the p110α mutant protein comprising a p110α helical domain mutant protein selected from the group consisting of a E545K, E542K, E545A, E545G or Q546K p110α helical domain mutant protein.

5. The method of claim 1, wherein the therapeutic agent binds to or complexes with a portion of IRS1 corresponding to AA 585-962.

6. The method of claim 1, the therapeutic agent comprising a stapled macrocyclic derivative of the polypeptide.

7. The method of claim 1, the polypeptide having an amino acid sequence selected from the group consisting of SEQ ID NOs: 1, 3, 4, 7 and 8.

8. The method of claim 1, the cancer cell comprising a solid tumor cell selected from the group consisting of a colon cancer cell, a rectal cancer cell, a lung cancer cell, a brain cancer cell, a head & neck cancer cell, a breast cancer cell, a skin cancer cell, a liver cancer cell, a pancreatic cancer cell, a stomach cancer cell, a uterine cancer cell, a cervical cancer cell, an ovarian cancer cell, a testicular cancer cell, a skin cancer cell or a esophageal cancer cell.

9. A method of treating cancer in a subject, the cancer including cancer cells expressing a p110α mutant protein, the method comprising:
administering to the cancer cells expressing the p110α mutant protein a therapeutically effective amount of an agent that inhibits binding of a p110α mutant protein to IRS1 in the cancer cells, the therapeutic agent comprising a polypeptide consisting of about 10 to about 40 amino acids, the polypeptide having at 80% sequence identity with consecutive amino acids of a portion of a helical domain of the p110α mutant protein that includes the mutated amino acid.

10. The method of claim 9, wherein inhibition of binding of the p110α mutant protein to IRS1 thereby inhibits the catalytic activity, signaling, and function of the p110α mutant protein in the cancer cell.

11. The method of claim 9, the amount of a therapeutic agent comprising the amount effective to inhibit proliferation, survival and/or motility of the cancer cell.

12. The method of claim 9, the p110α mutant protein comprising a p110α helical domain mutant protein selected from the group consisting of a E545K, E542K, E545A, E545G or Q546K p110α helical domain mutant protein.

13. The method of claim 9, the p110α mutant protein comprising a p110α E545K helical domain mutant protein.

14. The method of claim 9, wherein the therapeutic agent binds to or complexes with a portion of IRS1 corresponding to AA 585-962.

15. The method of claim 9, the therapeutic agent comprising a stapled macrocyclic derivative of the polypeptide.

16. The method of claim 9, the polypeptide having an amino acid sequence selected from the group consisting of SEQ ID NOs: 1, 3, 4, 7 and 8.

17. The method of claim 9, the cancer cell comprising a solid tumor cell selected from the group consisting of a colon cancer cell, a rectal cancer cell, a lung cancer cell, a brain cancer cell, a head & neck cancer cell, a breast cancer cell, a skin cancer cell, a liver cancer cell, a pancreatic cancer cell, a stomach cancer cell, a uterine cancer cell, a cervical cancer cell, an ovarian cancer cell, a testicular cancer cell, a skin cancer cell or a esophageal cancer cell.

18. A method of treating cancer in a subject, the cancer including cancer cells expressing a p110α mutant protein, the method comprising:
administering to the cancer cells expressing the p110α mutant protein a therapeutically effective amount of an agent that inhibits binding of a p110α mutant protein to IRS1 in the cancer cells, the therapeutic agent comprising a polypeptide consisting of about 10 to about 40 amino acids, the polypeptide having at 90% sequence identity with consecutive amino acids of a portion of a helical domain of the p110α mutant protein that includes the mutated amino acid selected from the group consisting of a E545K, E542K, E545A, E545G or Q546K.

19. The method of claim 18, the p110α mutant protein comprising a p110α E545K helical domain mutant protein.

20. The method of claim 18, wherein the therapeutic agent binds to or complexes with a portion of IRS1 corresponding to AA 585-962.

21. The method of claim 18, the therapeutic agent comprising a stapled macrocyclic derivative of the polypeptide.

22. The method of claim 18, the polypeptide having an amino acid sequence selected from the group consisting of SEQ ID NOs: 1, 3, 4, 7 and 8.
